(19) United States
(12) Patent Application Publication
(10) Pub. No.: US 2005/0027252 A1
(43) Pub. Date: Feb. 3, 2005

(54) Fluid removal apparatus for patient treatment

(76) Inventor: Alexander Boukas, Mattituck, NY (US)

Correspondence Address: Thomas A. O'Rourke;Bodner & O'Rourke, Suite 108, 425 Broadhollow Rd., Melville, NY, 11747, US

(21) Appl. No.: 10/632,134

(22) Filed: Jul. 31, 2003

(51) Int. Cl. A61M 1/00

(52) U.S. Cl. 604/118

(57) ABSTRACT

A fluid removal apparatus for patient treatment comprising a tube having a forward end and a rear end. The apparatus further comprises a body having at least one can of a compressed gas therein and an opening for releasing compressed gas. The tube can be attached to the opening in the body. The apparatus is operated by a trigger means attached to the body. The trigger means is adapted to cause gas to be released from the body thereby causing a suction effect at the forward end of the tube by regulating the flow of compressed gas from the cans of compressed gas out of the rear end of the tube.

### BACKGROUND OF THE INVENTION

Fluid build-up in the lungs can be a life-threatening condition, but it is often readily treatable. Fluid accumulation and swelling in the lungs, or pulmonary edema can arise for a number of reasons. One reason fluid in the lungs can arise is as a result of one of several cardiopulmonary afflictions. For example, pulmonary edema can be caused by heart failure that results in increased pressure in the pulmonary veins. The failing heart transmits its increased pressure due to blood build-up to the lung veins. As the pulmonary veins swell and become pressurized, fluid is pushed into the alveoli. The alveoli are the functional unit of the lungs, where gaseous exchange takes place. As more fluid is forced into the air spaces in the lungs, there becomes less space for gas exchange, the tidal volume decreases, and the person begins to have difficulty breathing.

Pulmonary edema has other origins associated with heart failure, such as myocardial infarction, leaking or narrowing of heart valves, or any condition whereby the heart muscle would weaken or stiffen. Each cause of pulmonary edema results in a build-up of pressure in the pulmonary veins due to an overload of fluid in the heart.

Pulmonary edema is usually treated with the administration of oxygen to the patient. For a serious case, intubation may be accompanied by mechanical respirations by a ventilator. Additionally, diuretics may be administered to accelerate water excretion from the body. However, while the pulmonary edema is corrected, the heart condition that caused it must be resolved as well.

Though pulmonary edema is usually the result of heart failure, fluid may accumulate in the lungs if a person ingests/inhales toxins, such as heat and poisonous gas. Severe infections, kidney failure and substantially harmful or deadly wounds may lead to fluid build-up in the lungs, as well.

Inhalation/ingestion of toxins, severe infection, and direct injury to the lungs are very prevalent in combat situation. For example, a gunshot wound to the chest may cause rapid accumulation of fluid in the lungs. Additionally, a severe wound may lead to an infectious condition, where fluid begins leaking into the air space causing difficulty in respiration.

In combat or severe trauma situations, clearing a patient's airway is usually the most critical step to begin treatment. Plasma, pleural fluid, saliva, blood, enrsis, secretions, broken teeth or other foreign bodies may be leak into the lungs or obstruct the patient's breathing. Thus, physicians, corpsman, and emergency response personnel need a portable suction device to quickly remove excess fluids in the patient's airway or airspace. Clearing fluid in the airway would also allow for easier intubation, if necessary.

Additionally, suctioning becomes a necessity in combat or emergency operations in non-hospital settings. For example, a field doctor may need to excise a bullet fragment and maintain a clear surgical field, a suctioning device will need to be employed. In this example, a suction device that is normally used in a hospital setting that operates by a bulky air compressor, that is not readily portable, would be undesirable. Thus, a lightweight, portable suctioning device would prove most useful.

Many devices have been developed that allow doctors to apply suction to fluids in the surgical field. Also, portable medical equipment has been engineered to allow emergency response personnel, field physicians, and armed forces doctors to effectively treat trauma patients and stabilize them for movement to a hospital.

One such suctioning device is taught in U.S. Pat. No. 4,490,138 to Lipsky, et al. The device in Lipsky is made for use in a hospital setting. The Lipsky patent teaches an attachment for a vacuum source used to remove fluids, tissue fragments, and debris from the surgical field. However, there is no suggestion that the device, which is an attachment, should be used outside of a hospital operating or emergency room.

A mobile suctioning device is taught in U.S. Pat. No. 4,930,997 to Bennett. The device in Bennett is sustained by a frame, which houses a motor, a pump, and a rechargeable battery. The Bennett device seems to be most useful in conjunction with an ambulance and its crew. The device, though portable, may be so only to-and-from an ambulance. A doctor traveling with his unit in a combat zone may not be able to carry a device with such a frame. Additionally, the use of a motor, pump and battery suggests that there are more sources for failure in the complicated device. The Bennett patent does not contain a suggestion for hand-held portability, nor for easy maintenance and cleaning should one of its parts fail during use.

A fluid-suctioning device is used in conjunction with a fluid delivering apparatus in U.S. Pat. No. 5,941,859 to Lerman. The Lerman device maintains a shield placed over a wound. While the shield is in place, high pressure fluid is delivered to the wound to irrigate and clean it. The suctioning portion simultaneously withdraws the high-pressure fluid and blood or bodily fluids. While the Lerman device can be used to irrigate a wound, it would not be used in a surgical situation requiring suction. The shield in the Lerman device does not allow a doctor to view or access the surgical field while irrigating and suctioning the area.

A nasal-nasopharyngeal cleaning system is described in U.S. Pat. No. 6,238,377 B1 to Lin. The device taught in Liu creates a suction effect to eliminate noxious or toxic gases that may have been inhaled. However, the Liu patent does not suggest that the device could be used orally or to clear the larynx of fluid or gas. Additionally, there is no suggestion that the device is portable, or can or should be used in emergency situations.

Suction pumps and emergency aspirators have been tested in various settings. The use of two hands and necessary instructional use have been seen as two shortcomings of prior art devices (see F. E. Amstein, A Practical Evaluation of Four Human-Powered Portable Airway Aspirators, 51 ANAESTHESIA 63, 68 (1996)). Also viewed as disadvantages of prior art aspirators have been high power requirements, low efficiency, use of a helper for operation of the device, and use of opaque tubing (see R. Rossi et al., Efficiency of Suction Pumps for the Emergency Medicine Setting, 9 ARCHIVES OF EMERGENCY MEDICINE 44, 44-50 (1992)). Furthermore, the need for intermittent testing and equipment checking are noted drawbacks that result from use of bulky and complex aspiration equipment (see Richard J. Kozak MD, et al., Difficulties with Portable Suction Equipment Used for Prehospital Advanced Airway Procedures, 1:2 PREHOSPITAL EMERGENCY CARE 91, 91-95 (April/June 1997)). The present device has been engineered in view of these disadvantages present in the prior art.

The disclosures and teachings of U.S. Pat. No. 6,094,778 to Boukas are herein incorporated by reference.

### OBJECTS OF THE INVENTION

It is an object of the present invention to provide a fluid removal apparatus for rapidly clearing fluid from an airway or wound site.

It is an object of the present invention to provide a fluid removal apparatus which can be held in a single hand and is human powered.

It is an object of the present invention to provide a fluid removal apparatus to allow for easier intubation.

It is an object of the present invention to provide a fluid removal apparatus which is portable, cost-effective and easy to maintain.

It is an object of the present invention to provide a fluid removal apparatus for removing excess fluids from a surgical field to allow doctors and pre-hospital care personnel to operate more effectively.

### SUMMARY OF THE INVENTION

The present invention is directed to biomedical applications of a fluid removal apparatus for patient treatment. The fluid removal apparatus is primarily useful for suctioning fluid or debris that is disposed in a patient's airway and removing excess fluid from a surgical field. The portability and sturdy design of the device increases its utility by allowing armed forces doctors and emergency response personnel to use a lightweight suctioning tool.

In one embodiment, the device comprises a tube attached to a body which houses one or more cans of compressed gas. The release valve of each can is connected to the tube via an export duct. A release means is attached at one end of the export duct, and at the other end to a trigger. When pressure is applied to the trigger, the release means pulls the export duct and opens the release valves on the cans. The compressed gas from the cans moves through the export duct into and out of a rear end of the tube. The flow of compressed gas causes lower pressure at the forward end of the tube. Accordingly, the forward end of the tube, a suction effect is created. The forward end of the tube allows for suitable attachments for various medical applications, such as elongated plastic tubes or rigid pipes. In this embodiment, the trigger is disposed on the bottom of the body, and the body is hand-held.

In another embodiment, the trigger is disposed on top of the body, and the body only houses one can of compressed air. When the trigger is depressed, the release valve feeds compressed air into the export duct. The export duct is connected to the tube, and the air flow creates a suction effect at the forward end of the tube.

In each embodiment, the forward end of the tube may be fitted with attachments, and the rear end of the tube may be fitted with attachments or a collecting pouch as desired.

In each embodiment, the cans of compressed gas may be encased in resistor wire. The resistor wire can be attached to a battery, which will cause the resistor wire to heat the cans of compressed gas. Heating the cans of compressed gas will increase the pressure of the gas inside the cans, and thus, maintain a constant suction effect. Alternatively, a phase change material (PCM) may be used to dissipate heat within the can. Heat expiration will cause the gas to expand, and it will maintain a constant suction effect.

### BRIEF DESCRIPTION OF THE DRAWINGS

FIG. 1 is a vertical cross-sectional view of a multiple canister embodiment of the present invention.

FIG. 2 is a vertical cross-sectional view of a single-canister embodiment of the present invention.

FIG. 3 is a front view of the device shown in FIG. 2.

FIG. 4 is a top view of the device shown in FIG. 2.

FIG. 5 is a rear perspective view of the device shown in FIG. 2.

FIG. 6 is a side view of a second embodiment of a single-canister version of the present invention.

FIG. 7 is a front view of the embodiment shown in FIG. 6.

FIG. 8 is a cross-sectional view of the embodiment shown in FIG. 6.

FIG. 9 is a rear perspective view of the embodiment shown in FIG. 6.

FIG. 10 is a top view of the embodiment shown in FIG. 6.

### DETAILED DESCRIPTION OF THE DRAWINGS

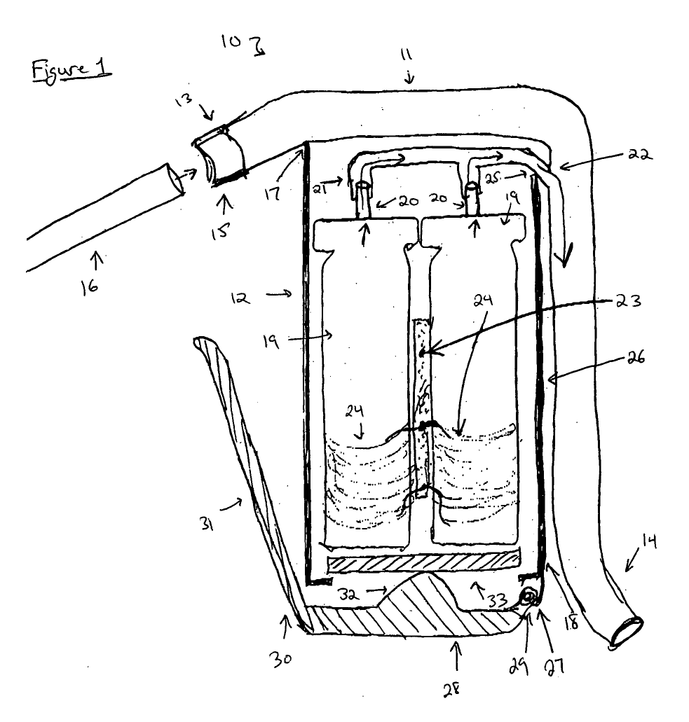

In FIG. 1, the fluid removal apparatus is generally shown at 10. A tube 11 may be any length. The tube may be made of any suitable material. One preferred material is a clear, flexible plastic. The tube may be attached to a body 12 by any suitable means. Examples of suitable means can include a hook and claw type material such as Velcro®, an adhesive or adhesive tape, or other means. The tube 11 has a forward end 13 and a rear end 14. On the forward end 13 of the tube 11 may be an attachment coupling 15. Various attachment couplings are known in the art. In this example, the coupling can be a ring or sleeve that creates a frictional fit between the inner surface of the sleeve or ring and the outer surface of the tube. Alternatively, any suitable and adhesive material can be used. When needed, an attachment 16 may be fitted to the forward end 13 of tube 11. In one example, the attachment can have a tube portion that is set into one end of the coupling. The coupling 15 will provide a vacuum seal between the tube and the attachment.

The coupling may be made of any suitable material, such as metal, plastic or rubber. Tube 11 can be fixedly or removably attached to body 12. Tube 11 may be made of any suitable material for handling gases, liquids and small solids. As seen in FIG. 1, tube 11 can be attached to the top surface 17 and/or side surface 18 of body 12. However, the tube 11 need only be attached to the body 12 while the device is being used. For example, when cleaning or replacing the tube, it may be removed from the body.

The body 12 is preferably substantially hollow and houses one or more cans of compressed gas 19. In one preferred embodiment, there may be multiple cans of compressed gas. Each can of gas may be removable for ready replacement, if desired. Alternatively, the device may be a single use device that can be disposed of, if desired. Each can of gas 19 has a release valve 20, which are all connected to the tube 11 by an export duct 21. When activated, the release valves feed compressed gas into the export duct, which passes into tube 11 at inlet or port 22. The export duct may be made of any suitable tubing material for handling a gaseous material.

Between the cans of compressed gas 19 can be a battery 23. The battery 23 may be connected to resister wire 24 which is wrapped around each can. When activated, the current in the resister wire heats the cans to increase the pressure of the gas inside the cans and thus increase the suction created. Heat sensitive fuse blocks (not shown) may be employed to prevent overheating.

In another embodiment, the battery and resistor wire may be eliminated. In this embodiment, a PCM may be used in the can of compressed gas. The PCM should be a material that is capable of latent heat storage. A PCM is a material that will stay at relatively the same temperature during phase change. For example, PCM's absorb and retain heat when changing from solid to liquid, but release heat when changing from liquid to solid. Some PCMs that can be used with the can of compressed gas are paraffin waxes, normal paraffms and Fischer-Tropsch hard waxes. Preferably, the PCMs have a melting point between about -3 degrees Celsius and 100 degrees Celsius. When compressed gas is expelled from the can and the temperature decreases, the PCM will release heat, which may keep the suction effect constant.

An activation means 26 runs along side surface 18 of body 12. At one end 25, the activation means 26 may be in the vicinity of or attached to export duct 21. At a second end 27, the activation means 26 may be attached to a first end 29 of a trigger 28. At a second end 30 of trigger 28 is a depression means 31. Depression means 31 may be molded as a hand-grip, with finger indentations to allow the user to grip and squeeze easily. The trigger 28 is operated as a lever with fulcrum 32 resting, or hingedly attached, to the bottom surface 33 of body 12.

In operation, the user may wrap his/her hand around the tube, body and trigger, or alternatively, around the body and trigger leaving the tube to hang over the back of the hand. As the depression means 31 is squeezed toward the body, activation means 26 pulls export duct 21 thereby opening the duct. When the export duct 21 is pulled and opened, the release valves 20 allow compressed gas to escape. The compressed air flows through the export duct 21 and out of the rear end 14 of tube 11. The rapid flow of compressed gas out of the rear end of tube 11 creates a partial vacuum or suction effect at the forward end 13 of tube 11. As the depression means 31 is squeezed closer to the body 12, more compressed gas is released from the rear end 14 of tube 11, creating a greater suction effect. Thus, the user can temper the amount of suction from lightly suctioning toxic gas to removing debris particles by applying different amounts of pressure to depression means 31. The lightweightedness, hand-held operation, and manual operability make this device an optimum tool for field medics and emergency personnel.

FIG. 2 depicts another embodiment of the present invention at 101. In this embodiment, only a single can of compressed gas 102 is preferably utilized. While the tube 103 placement is similar to the previous embodiment, the release of compressed gas differs.

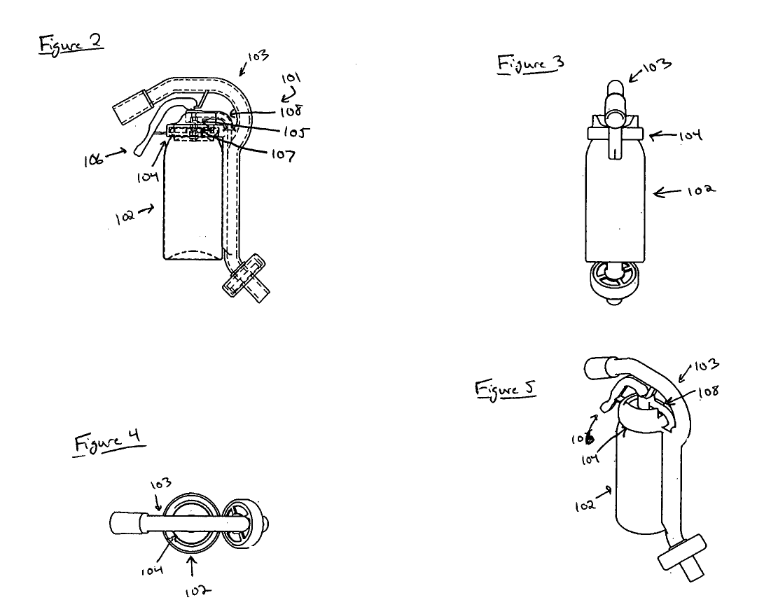

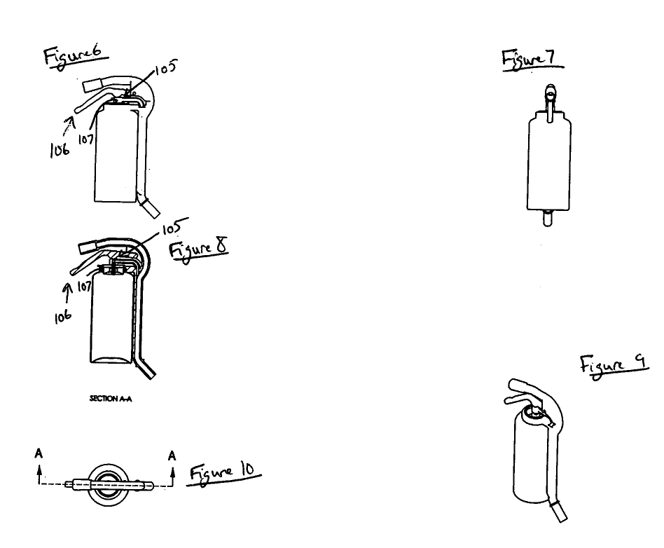

The device 101 attaches to the can of compressed gas 102 by an airtight cap 104. The cap 104 encapsulates the release valve 105 on the top of the can of compressed gas. In this embodiment, a trigger 106 can be adapted to operate the release valve 105. In one embodiment, as seen in FIGS. 2-5, the trigger 106 can attach to the release valve 105. In another embodiment, as seen in FIGS. 6-10, the trigger 106 can rest on the release valve 105. Hence, when the trigger 106 is depressed, by a forefinger in either embodiment, the release valve 105 is opened into an airtight pocket 107. The pocket 107 maintains the airtight environment from the can and forces the compressed gas out of an export duct 108 into tube 103. As the air is forced out of the tube 103, a suction effect is created. The amount of suction can be controlled by the amount of pressured placed on the trigger 106.

In this embodiment, a battery (not shown) may be connected to resister wire, which may be wrapped around the can of compressed gas 102. When activated, the current in the resister wire heats the cans to increase the pressure of the gas inside the cans and thus increase the suction created. Heat sensitive fuse blocks (not shown) may be employed to prevent overheating. Additionally, the resistor wire (not shown) and can 102 may be covered by an insulating material (not shown). The insulating material will effectively contain the heat created by the activation of the resistor wire. Also, the insulating material will protect the user's hand from any uncomfortable temperature changes that may be associated with activation of the battery. Also, in this embodiment, a PCM may be used in substantially the same manner as described above.

It is understood that the body, tube and can of compressed gas may be of any size, shape or material. Preferably, the tube will be a soft plastic and body will be hard plastic for easy maintenance and cleaning. Additionally, the body may have a clip for attachment to a belt or strap of a bag. Also, the cans of compressed gas will be easily replaceable in the present invention, by either removing them from the body, or removing the cap.

In any embodiment, a container (not shown) may be attached to the rear end 14 of the tube 11. Preferably, the container is a bag or pouch that will enable the user to collect any fluid that is suctioned through tube 11. The container may have an open end, which will preferably create an airtight seal around the rear end 14 of tube 11. In some manner, the bag may allow gas to escape, while suctioning liquid, debris or polluted air. In one embodiment, the container will be equipped with an attachment port, which can mate with a filter. The filter may allow gas to escape, while retaining liquid, debris or polluted gas. In a second embodiment, the container itself may be manufactured from a gas-permeable, liquid-retaining compound. In a further embodiment, the container may be equipped with vents that will allow gas to escape. In each embodiment, the container may be equipped with a sealing means, which will allow the user to remove and seal the container. A sealing means, which may be an adhesive strip or cap, can be used if the container houses hazardous material, or a liquid that can be reused after being filtered.

1. A fluid removal apparatus for patient treatment comprising:
a tube having a forward end and a rear end;
a body having at least one can of a compressed gas therein and an opening for releasing said compressed gas, said tube being attached to said opening in said body; and
a trigger means attached to said body, and said trigger means adapted to cause said gas to be released from said body thereby causing a suction effect at said forward end of said tube by regulating the flow of compressed gas from said cans of compressed gas out of said rear end of said tube.

2. A fluid removal apparatus according to claim 1, which said forward end of said tube is fitted with a coupling for securing an attachment thereto.

3. A fluid removal apparatus according to claim 1, wherein there are two or more cans of compressed gas.

4. A fluid removal apparatus according to claim 4, wherein said cans are connected by an export duct.

5. A fluid removal apparatus according to claim 1, wherein said body has a battery, said battery connected to resister wire, said resister wire being wrapped around said can of compressed gas.

6. A fluid removal apparatus according to claim 5, wherein said body has heat sensitive fuse blocks to prevent overheating.

7. A fluid removal apparatus for patient treatment device comprising:
a tube having a forward end and a rear end;
a cap attaching said tube to a can of compressed gas; and,
a trigger attached to said cap, said trigger adapted to control a suction effect at said forward end of said tube by regulating the flow of compressed gas from said cans of compressed gas out of said rear end of said tube.

8. A fluid removal apparatus according to claim 7 wherein said forward end of said tube is fitted with a coupling for an attachment.

9. A fluid removal apparatus according to claim 7 wherein said cap makes an airtight seal with said can of compressed gas.

10. A fluid removal apparatus according to claim 7 wherein said can of compressed gas has a release valve.

11. A fluid removal apparatus according to claim 10 wherein said release valve is controlled by said trigger.

12. A fluid removal apparatus according to claim 7 wherein said can of compressed gas is connected to said tube by an export duct.

13. A hand-held, self-contained fluid removal apparatus comprising:
a tube having a forward end and a rear end;
a cap attaching said tube to a can of compressed gas; a trigger attached to said cap, said trigger adapted to control a suction effect at said forward end of said tube by regulating the flow of compressed gas from said cans of compressed gas out of said rear end of said tube; and,
a container attached to said rear end of said tube.

14. A container for receiving gases, fluids, and solids comprising:
an open end;
an attachment port on a surface of said container; and,
a filter removably attached to said attachment port, said filter being adapted to allow gas to escape while inhibiting the release of liquid, polluted gas and solids.

15. A container according to claim 14 wherein said open end is adapted to create an airtight seal.

16. A container according to claim 14 wherein said open end has a sealing means which allows the container to be sealed airtightly.

17. A container according to claim 16 wherein said sealing means is an adhesive strip.

18. A container according to claim 16 wherein said sealing means is a cap adapted to fit airtightly over said open end of said container.

19. A method of removing fluids, gases, or solids from the airway or wound site of a patient comprising:
positioning a tube in said airway or said wound site, said tube being attached to a fluid removal apparatus which comprises a tube having a forward end and a rear end, a cap attaching said tube to a can of compressed gas, and a trigger attached to said cap, said trigger adapted to control a suction effect at said forward end of said tube by regulating the flow of compressed gas from said cans of compressed gas out of said rear end of said tube;
applying pressure to said trigger to create a suction effect at said front end of said tube; and,
suctioning liquids, gases, or solids from said airway or said wound site.

20. A method of removing fluids, gases, or solids from the airway or wound site of a patient comprising:
positioning a tube in said airway or said wound site, said tube being attached to a fluid removal apparatus which comprises a tube having a forward end and a rear end, a cap attaching said tube to a can of compressed gas, a trigger attached to said cap, said trigger adapted to control a suction effect at said forward end of said tube by regulating the flow of compressed gas from said cans of compressed gas out of said rear end of said tube, and a container attached to said rear end of said tube;
applying pressure to said trigger to create a suction effect at said front end of said tube; and,
suctioning liquids, gases, or solids from said airway or said wound site.
